(19) United States
(12) Patent Application Publication
(10) Pub. No.: US 2009/0018199 A1
(43) Pub. Date: Jan. 15, 2009

(54) Methods of Administering 3, 3, 14, 14 Tetramethyl Hexadecane 1, 16 Dioic Acid

(75) Inventors: Jacob Bar-Tana, Jerusalem (IL); Ihor Bekersky, Antioch, IL (US)

Correspondence Address: COOPER & DUNHAM, LLP, 1185 AVENUE OF THE AMERICAS, NEW YORK, NY, 10036, US

(73) Assignee: SYNDROME X LTD., Jerusalem (IL)

(21) Appl. No.: 10/585,017

(22) PCT Filed: Dec. 30, 2004
(86) PCT No.: PCT/IL2004/001185

§ 371 Date: Aug. 4, 2008

Related U.S. Application Data
(60) Provisional application No. 60/533,639, filed on Dec. 30, 2003.

(51) Int. Cl. A61K 31/194; A61P 3/00

(52) U.S. Cl. 514/574

(57) ABSTRACT

The present invention provides methods for lowering LDL, VLDL, total cholesterol, triglycerides, Insulin resistance and hypertension, and methods for elevating HDL in subjects in need thereof. Additionally, the present invention provides methods of administering 3,3,14,14 tetramethyl hexadecane 1,16 dioic acid for the above indications.

This application claims priority of U.S. Provisional patent application No. 60,533,639 which is hereby incorporated by reference in its entirety.

### FIELD OF THE INVENTION

The present invention relates to methods of treating dyslipidemia, elevated triglycerides, low HDL cholesterol, high LDL and/or VLDL cholesterol, hypertension, and other morbidities which may be associated with Metabolic Syndrome (Syndrome X) or other diseases.

### BACKGROUND OF THE INVENTION

Lowering of serum lipids is increasingly recognized as essential in the prevention of atherosclerotic disease. In the last decade there has been increasing identification of people whose serum cholesterol, triglycerides, and HDL-cholesterol require clinical assessment and, frequently, treatment.

Meta-analyses of prospective studies indicate that elevated triglycerides also are an independent risk factor for coronary artery disease (CAD). Moreover, it has been appreciated that hypertriglyceridemia increases the risk of atherosclerosis to a higher risk than that which would be predicted from the cholesterol concentration only.

The Adult Treatment Panel III (ATPIII) guidelines of the National Cholesterol Education Program (NCEP) identifies lowering elevated triglycerides as a therapeutic goal. Its recommendations include decreasing triglycerides levels, and reduction of VLDL as well as LDL in persons with elevated triglycerides. Elevated triglycerides are identified in the International Classification of Diseases, Ninth Revision as part of diagnostic code (ICD-9-CM) 277.7.

Currently existing hypolipidemic drugs include HMG-CoA reductase inhibitors, niacin, bile acid-binding resins and fibric acid derivatives.

### HMG-CoA Reductase Inhibitors (Statins)

This class of drugs, which include lovastatin, simvastatin, pravastatin, fluvastatin, atorvastatin, and cerivastatin, inhibits the rate-limiting step in hepatic cholesterol biosynthesis (the conversion of HMG-CoA to mevalonate), causing an increase in LDL receptor levels in hepatocytes and enhanced receptor-mediated clearance of LDL cholesterol from the circulation. At usual doses, the HMG-CoA reductase inhibitors decrease total cholesterol by 20 to 30% and LDL cholesterol by 25 to 40%. Larger reductions may be achieved with higher doses. Treatment with reductase inhibitors often reduces triglycerides by 10 to 20%, possibly due to reduced secretion of VLDL by the liver. Higher doses of more potent reductase inhibitors, which can lower LDL cholesterol by 45 to 60%, can lower triglycerides by 30 to 45%. HDL cholesterol levels rise about 5 to 10%. In comparison with other lipid-lowering agents, HMG-CoA reductase inhibitors are relatively free of side effects. Mild, transient elevation of liver enzymes occur with all of the agents at the highest doses, but elevation of serum aminotransferases, to more than three times the upper limits of normal, occurs in <2% of patients. Therapy should be discontinued when elevations of this magnitude occur. A rare but potentially serious adverse effect of HMG-CoA reductase inhibitors is myopathy, manifest by muscle pain with elevation of serum creatine phosphokinase (CPK). This occurs in <1% of patients treated with reductase inhibitors alone but is more common (about 2 to 3%) when used in combination with gemfibrozil, niacin, or cyclosporine.

### Niacin

The mechanism of action of niacin is not fully understood, but it appears to inhibit the secretion of lipoproteins containing apo B100 from the liver. Niacin decreases both total and LDL cholesterol approximately 15 to 25%, reduces VLDL levels by 25 to 35%, and raises HDL cholesterol levels by as much as 15 to 25%. Thus, niacin exerts favorable changes on the three major lipoproteins (VLDL, LDL, and HDL). Efficacy of monotherapy was confirmed in a long-term secondary prevention trial in which niacin significantly reduced the incidence of myocardial infarction. An even longer-term follow-up of that study (15 years total) showed an 11% decrease in all-cause mortality among patients randomized to niacin. Because of its ability to reduce VLDL synthesis, niacin is also a first-line drug for treatment of hypertriglyceridemia.

Niacin is safe, having been in use for almost 30 years, but unpleasant side effects, including cutaneous flushing with or without pruritus, may limit patient acceptability. The cutaneous symptoms tend to subside after several weeks and may be minimized by initiating therapy at low doses or by administering aspirin 30 min before the niacin dose. Less common adverse effects include elevation of liver enzymes, gastrointestinal distress, impaired glucose tolerance, and elevated serum uric acid levels, with or without gouty arthritis. Liver enzymes may be elevated in 3 to 5% of patients on full doses of niacin (>2 g/d). Because of its propensity to worsen the control of blood sugar, niacin should be used with caution in patients with diabetes. Niaspan, an intermediate-release form of niacin, appears to exhibit lipid-altering activity similar to regular niacin.

### Bile Acid-Binding Resins

Cholestyramine and colestipol have been in use as lipid-lowering agents for almost three decades. These drugs interfere with reabsorption of bile acids in the intestine, resulting in a compensatory increase in bile acid synthesis and upregulation of LDL receptors in hepatocytes. The bile acid sequestrants are useful in the treatment of patients with elevated levels of LDL cholesterol and normal triglycerides. Sequestrants produce dose-dependent decreases in the order of 15 to 25% in total cholesterol and of 20 to 35% in LDL cholesterol. The agents cause modest increases in HDL cholesterol. A limitation of the sequestrants is their tendency to raise triglyceride levels through compensatory increases in hepatic synthesis of VLDL; they should not be given to hypertriglyceridemic individuals. Bile acid-binding resins are efficacious and safe and are recommended for young adult men and premenopausal women with moderate cholesterol elevations. Patient compliance is low, in part because of the need to dissolve these powdered agents in fluid; the availability of colestipol as a tablet may alleviate this problem. Gastrointestinal side effects include constipation, bloating, and gas.

### Fibric Acids

Gemfibrozil and fenofibrate reduce VLDL triglyceride entry into plasma and reduce synthesis of apo CIII, which might improve LPL (lipoprotein lipase)-induced lipolysis or reduce VLDL secretion. Stimulation of peroxisomal fatty acid oxidation by fibrates may also contribute to the triglyceride-lowering actions. Gemfibrozil and fenofibrate treatment is associated with 25 to 40% reductions in plasma triglyceride levels. Postprandial triglyceride levels, which are linked to fasting concentrations, are also reduced. HDL cholesterol levels increase 5 to 15% with fibrate treatment. Fibric acids and a low-fat diet are particularly useful in the treatment of dysbetalipoproteinemia and are first-line therapy for this disorder except in postmenopausal women, who should initially be given estrogen replacement (if not contraindicated).

Significant increases in LDL cholesterol can accompany otherwise potentially beneficial falls in triglycerides and increases in HDL cholesterol during fibrate therapy. Such rises may require a change to another drug or addition of a second agent.

In the short term, these drugs are well tolerated; mild gastrointestinal distress in the form of epigastric pain is the major side effect. Elevation of liver enzymes occurs in 2 to 3% of patients but does not usually require cessation of treatment. Rarely, hepatitis can occur. Fibrates appear to make the bile more lithogenic, and long-term use is probably associated with a twofold increase in gallstone formation. Myopathy with myositis is a rare occurrence with the fibrates when given alone. (See Harrison's Principles of Internal Medicine (various editors), 15^{th} edition, McGraw-Hill (2001).

In clinical practice, elevated triglycerides are most often observed in persons with. Metabolic Syndrome (also known as Syndrome X). Risk factors for elevated triglycerides are life style factors (physical inactivity, smoking, excess alcohol intake, high carbohydrate diets), several diseases (diabetes, renal failure, nephrotic syndrome), or certain drugs. Hypertriglyceridemia is often caused by genetic factors, the most common of which is familial combined hyperlipidemia, which occurs in 1:50 in the general population.

The medical and scientific communities have become progressively aware of the etiological-pathophysiological linkage between the combined pathologies that make up Metabolic Syndrome. Insulin resistance, atherogenic dyslipoproteinemia, abdominal obesity, raised blood pressure and prothrombic and proinflammatory states are viewed as reflections of a unifying syndrome leading to atherosclerotic cardiovascular disease.

NCEP ATPIII (see above) determines the clinical identification of an individual suffering from Metabolic Syndrome as one having at least 3 of the following criteria:

| Risk Factor | Defining Level | |
|---|---|---|
| Abdominal obesity | Waist circumference | |
| Men | >102 | cm (>40 in) |
| Women | >88 | cm (>35 in) |
| Triglycerides | ≧150 | mg/dL |
| HDL cholesterol | | |
| Men | <40 | mg/dL |
| Women | <50 | mg/dL |
| Blood pressure | ≧130/≧85 | mmHg |
| Fasting glucose | ≧110 | mg/dL |

Identification and clinical management of Metabolic Syndrome can prevent or ameliorate type 2 diabetes and cardiovascular disease. The management should focus on therapeutic lifestyle changes (TLC), LDL reduction, and overall optimization of the lipid profile. A more comprehensive pharmacological approach is desirable, as no drug designed along the etiological-pathological principles of Metabolic Syndrome is currently available.

Based on in vitro studies and studies in animal models, Medica 16 is a potent, hypolipidemic, calorigenic, antidiabetogenic compound particularly well suited for the treatment and prevention of dyslipoproteinemia (combined hypercholesterolemia-hypertriglyceridemia, low HDL-cholesterol), obesity, and impaired glucose tolerance (IGT) leading to NIDDM, Medica 16 may prove as the drug of choice for Metabolic Syndrome patients (see Bar-Tana J, Kahn-Rose G, Srebnik B. Inhibition of lipid synthesis by beta, beta tetramethyl-substituted C14-C22 alpha, dicarboxylic acids in the rat in vivo. J Biol Chem 1985; 260: 8404-8410.; Rose-Kahn G, Bar-Tana J. Inhibition of lipid synthesis by beta, beta′-tetramethyl-substituted, C14-C22, alpha, omega-dicarboxylic acids in cultured rat hepatocytes. J Biol Chem 1985; 260: 8411-8415.; Bar-Tana J, Rose-Kahn G, Frenkel B, Shafer Z, Fainaru M. Hypolipidemic effect of beta, beta′-methyl-substituted hexadecanedioic acid (Medica 16) in normal and nephritic rats. J Lipid Res 1988; 29: 431-441.; Frenkel B, Mayorek N, Hertz R, Bar-Tana J. The hypochylomicronemic effect of beta, beta′-methyl-substituted hexadecanedioic acid (Medica 16) is mediated by a decrease in apolipoprotein C-m. J Biol Chem 1988; 263: 8491-8497.; Tzur R, Rose-Kahn G, Bar-Tana J. Hypolipidemic, antiobesity, and hypoglycemic-hypoinsulinemic effects of beta, beta′-methyl-substituted hexadecanedioic acid in sand rats. Diabetes 1988; 37: 1618-1624.; Tzur R, Smith E, Bar-Tana J. Adipose reduction by beta, beta′-tetramethyl-substituted hexadecanedioic acid (Medica 16). Int J Obes 1989; 13: 313-326.; Bar-Tana J, Ben-Shoshan S, Blum J, Migron Y, Hertz R, Pill J, Rose-Kahn G, Witte E C. Synthesis and hypolipidemic and antidiabetogenic activities of beta, beta′, beta′-tetra substituted, long-chain dioic acids. J Med Chem 1989; 32: 2072-2084.; Mayorek N, Kalderon B, Itach E, Bar-Tana J. Sensitization to insulin induced by beta, beta′-methyl-substituted haxadecanedioic acid (Medica 16) in obese Zucker rats in vivo. Diabetes 1997; 46(12): 1958-1964.; Kalderon B, Mayorek N, Ben-Yaakov L, Bar-Tana J. Adipose tissue sensitization to insulin induced by troglitazone and Medica 16 in obese Zucker rats in vivo. Am J Physiol Endocrine Metab 2003 (In Press)). The hypolipidemic effect of Medica 16 is characterized by a pronounced decrease in plasma triglycerides and cholesterol in normolipidemic animals and normalization of plasma lipids in hyperlipidemic animal models. The hypolipidemic effect is due to a pronounced inhibition of liver very low-density lipoproteins (VLDL) synthesis together with activation of the clearance of plasma chylomicrons and VLDL. Inhibition of liver VLDL synthesis is due to suppression of liver microsomal triglycerides transfer protein (MTP). Activation of clearance is secondary to suppression of liver apolipoprotein CIII synthesis and consequent disinhibition of lipoprotein lipase. Suppression of liver MTP and apolipoprotein CIII is due to transcriptional suppression of liver hepatocyte nuclear-factor-4α (HNF-4α), independently of PPARα activation. Increased plasma HDL-cholesterol is observed secondary to normalization of plasma triglycerides. Medica 16 may thus prove to be a valuable option for the treatment of combined hyper-cholesterolemia-hypertriglyceridemia or in isolated hypertriglyceridemia with low plasma HDL-cholesterol or for lowering postprandial plasma chylomicrons (see Bar-Tana J. The hypolipidemic effect of beta, beta′-tetramethyl hexadecanedioic acid (Medica 16) in hyperlipidemic JCR: LA-corpulent rats. Arteriosclerosis and Thrombosis 1991; 11: 602-609.; Kalderon B, Hertz R, Bar-Tana J.

Tissue selective modulation of redox and phosphate potentials by beta, beta′-methyl-substituted hexadecanedioic acid. Endocrinology 1992; 131: 400-407.; Mayorek N, Bar-Tana J. Hypocheolesterolemic effect of beta, beta′-methyl-substituted hexadecanedioic acid (Medica 16) in the male hamster. Biochem J 1993; 289(Pt 3): 911-917.; Frenkel B, Bishara-Shieban J, Bar-Tana J. The effect of beta, beta′-tetramethyldecanedioic acid (Medica 16) on plasma very-low-density lipoprotein metabolism in rats: role of apolipoprotein C-III. Biochem J 1994; 298(Pt 2): 409-414.; Atkinson L, Kelly SE, Russel J C, Bar-Tana J, Lopaschuk G D. Medica 16 inhibits hepatic acetyl-CoA carboxylase and reduces plasma triacylglycerol levels in insulin-resistant JCR: LA-cp rats. Diabetes 2002; 151: 1548-1555.).

### M16 (Medica 16) Development

Medica 16, referred to henceforth as M16, 3,3,14,14-tetramethylhexadecane-1,16-dicarboxylic acid, is a β,β′-methyl substituted α,ω-dicarboxylic acid of sixteen carbons in chain length. M16 is prepared essentially as described in U.S. Pat. No. 4,634,795. The structure of M16 is:

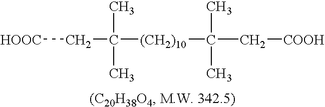

### Non-Clinical Pharmacology

As described above, M16 was found to be a potent hypolipidemic, antidiabetogenic, and calorigenic compound well suited for the treatment and prevention of dyslipoproteinemia (combined hypercholesterolemia-hypertriglyceridemia, low HDL-cholesterol), obesity, and impaired glucose tolerance (IGT) leading to NIDDM. The hypolipidemic effect of M16 is characterized by a pronounced decrease in plasma triglycerides and cholesterol in normolipidemic animals and normalization of plasma lipids in hyperlipidemic animal models. (see above references). The hypolipidemic effect is due to a pronounced activation of clearance of plasma chylomicrons and very-low-density lipoproteins secondary to inhibition of apolipoprotein CIII synthesis and consequent disinhibition of lipoprotein lipase activity and hepatic lipase activities. Increased plasma HDL-cholesterol is observed secondary to normalization of plasma triglycerides. Medica 16 may thus prove to be a valuable option for the treatment of combined hypercholesterolemia-hypertriglyceridemia or for treating isolated hypertriglyceridemia with low plasma HDL-cholesterol or for lowering postprandial plasma chylomicrons.

### Safety Pharmacology

The safety pharmacology of M16 and reference compounds was evaluated in 85 different tests in animals. The acute toxicity as well as activity in the central nervous system, cardiovascular system, metabolic and endocrine systems, inflammation, allergy, immunopharmacology, gastrointestinal system, and antimicrobial activity were evaluated. A number of non-categorized tests were also performed including receptor agonist/antagonist (histamine, substance P, anticholinergic, adrenergic, estrogen, thromboxane, antiserotonin, 5-HTP) potentiation.

Other than a slight antihypertensive effect observed in spontaneously hypertensive rats at an oral dose of 100 mg/kg, no activity was noted.

At 400 mg/kg given orally to rats or dogs, no hemodynamic effects were observed. No effects on urine and electrolyte excretion were found after oral administration of M16 at 400 mg/kg.

### Non-Clinical Toxicology

In an acute toxicity study in mice, the oral lethal dose was estimated as >2250 mg/kg. In a 4-week oral toxicity study in rats at doses of 100, 400, and 1600 mg/kg/day, the only effects found were at 1600 mg/kg: slight increase in ALT (females), slight increases in alkaline phosphatase (males and females), slight increase in leukocytes (males), and increase in liver weight in both sexes due to hepatic peroxisome proliferation. The NOAEL dose was considered to be 400 mg/kg/day. In a 4-week toxicity study in dogs at 50, 200, and 800 mg/kg/day, no effects were noted; the NOAEL was at least 800 mg/kg/day. M16 was negative in various genotoxicity assays.

### Non-Clinical ADME

M16 is absorbed via the GI-portal pathway. The terminal elimination half-life (t/_{1/2}) of M16 in rats (3.1 hr) is longer than the time reported (1.2 hr) for other hypolipidemic drugs (i.e., fibrates). This is reflected in the relatively lower clearance and larger volume of distribution relative to the fibrates. Absorption of M16 was relatively rapid (Tₘₐₓ1.6 hr) and was essentially quantitative after administration of the 200 mg oral dose. After administration of ³H-M16, the highest concentrations of total radioactivity were found in the following order: liver>small intestine>plasma>peripheral and epididymal fat. Other tissues examined (i.e., heart, muscle, lung) had low and insignificant concentrations of total radioactivity.

Further information regarding Medica 16 in connection with the above mentioned indications can be found in U.S. Pat. Nos. 4,634,795, 4,689,344, 4,711,896, 5,641,810, 6,284,903, and 6,303,653 and in PCT publication Nos. WO 98/30530 and WO 99/00116 which are hereby incorporated by reference in their entirety.

### SUMMARY OF THE INVENTION

In some aspects, the present invention provides methods for elevating the plasma level of HDL cholesterol in a human subject in need thereof.

In other aspects, the present invention provides methods for decreasing the plasma level of LDL cholesterol in a human subject in need thereof.

In additional aspects, the present invention provides methods for decreasing the plasma level of triglycerides in a human subject in need thereof.

Yet additional aspects provide methods of decreasing the plasma level of VLDL cholesterol in a human subject in need thereof.

Further, methods of decreasing the plasma level of total cholesterol in a human subject in need thereof are provided.

Additionally, methods for decreasing insulin resistance and hypertension in a human subject in need thereof are provided.

Said methods comprise periodically orally administering to the human subject 3,3,14,14 tetramethyl hexadecane 1,16 dioic acid in various dosage regimens, as indicated herein.

### DETAILED DESCRIPTION OF THE INVENTION

Three clinical studies of M16 were performed: a Phase 1 single- and repeated-dose pharmacokinetic-tolerance study in normal male volunteers; a pilot repeated-dose safety and efficacy of up to 4 months of dosing in obese, dyslipoproteinemic, nondiabetic male subjects; and a pilot efficacy study for up to 32 weeks in obese, dyslipoproteinemic, insulin resistant male subjects. These studies are described in Examples 1-3.

Based on the pharmacokinetic results, M16 was concluded to be highly safe and non-toxic. A striking effect of M16 was observed in the second trial within one month, where it reduced triglycerides (baseline range 380-1156 mg %) by 44% and reduced total cholesterol (baseline range 185-346 mg %) by 14% at 200 mg, with further reductions by 59% and 30% compared with baseline, respectively, at 300 mg.

All clinical chemistries, hematology, ECGs, and urinalyses were normal in all study subjects at all doses administered.

Based on preliminary results, the inventors of the instant application have found that a particular dosage range is highly effective with regards to the patients' response to treatment, as measured by various clinical endpoints. Doses above said range may likely not increase efficacy, and in some cases may even decrease efficacy (depending on the individual patient, indication to be treated and other factors) while they do increase the exposure of the patient and, over time, may result in undesirable side-effects stemming from high prolonged exposure.

Thus, in one aspect the present invention concerns a method for treatment of a symptom associated with Metabolic Syndrome in a human subject in need thereof comprising periodically orally administering to the human subject 3,3,14,14 tetramethyl hexadecane 1,16 dioic acid in an amount from about 30 mg per day to about 800 mg per day.

Additionally, the present invention provides a method for elevating the plasma level of HDL cholesterol in a human subject in need thereof comprising periodically orally administering to the human subject 3,3,14,14 tetramethyl hexadecane 1,16 dioic acid in a range from about 30 mg per day to about 800 mg per day. The plasma level of HDL cholesterol may be elevated by at least 5%, at least 10%, at least 15%, at least 20%, at least 25% or even at least 30% or 35% as compared to the level prior to treatment. Additionally, The plasma level of HDL cholesterol may be elevated above at least 30 or 40 mg/DL. Further, the method may comprise maintaining the plasma level of HDL cholesterol above the level prior to the treatment by the percentages described above and/or above 30 or 40 mg/DL.

The present invention further provides a method for decreasing the plasma level of LDL cholesterol in a human subject in need thereof comprising periodically orally administering to the human subject 3,3,14,14 tetramethyl hexadecane 1,16 dioic acid in a range from about 30 mg per day to about 800 mg per day. The plasma level of LDL cholesterol may decrease by at least 5%, at least 10%, at least 15%, at least 20%, at least 25%, at least 30%, at least 40%, at least 50% or even at least 55 or 60% as compared to the level prior to treatment. Additionally, the plasma level of LDL cholesterol may be decreased below at least 190 mg/DL, at least 160 mg/DL, at least 130 mg/DL or even at least 100 mg/DL. Further, the method may comprise maintaining the plasma level of LDL cholesterol below the level prior to the treatment by the percentages described above and/or below the values described above.

Further, the present invention provides a method for decreasing the plasma level of VLDL cholesterol in a human subject in need thereof comprising periodically orally administering to the human subject 3,3,14,14 tetramethyl hexadecane 1,16 dioic acid in a range from about 30 mg per day to about 800 mg per day. The plasma level of VLDL cholesterol may decrease by at least 5%, at least 10%, at least 20%, at least 25%, or even at least 30% or 35% as compared to the level prior to treatment. Further, the method may comprise maintaining the plasma level of VLDL cholesterol below the level prior to the treatment by these percentages.

Additionally, the present invention provides a method for decreasing the plasma level of cholesterol in a human subject in need thereof comprising periodically orally administering to the human subject 3,3,14,14 tetramethyl hexadecane 1,16 dioic acid in a range from about 30 mg per day to about 800 mg per day. The plasma level of cholesterol may decrease by at least 10%, at least 15%, at least 20%, at least 25%, at least 30%, at least 40%, at least 50% or even at least 55 or 60% as compared to the level prior to treatment. Additionally, the plasma level of cholesterol may be decreased below at least 240 mg/DL or at least 200 mg/DL. Further, the method may comprise maintaining the plasma level of cholesterol below the level prior to the treatment by the percentages described above and/or below the values described above.

In addition, a method for decreasing the plasma level of triglycerides in a human subject in need thereof is provided, said method comprising periodically orally administering to a human subject 3,3,14,14 tetramethyl hexadecane 1,16 dioic acid in a range from about 30 mg per day to about 800 mg per day. The plasma level of triglycerides may decrease by at least 7%, at least 10%, at least 15%, at least 20%, at least 25%, at least 30%, at least 40%, at least 50% or even at least 55 or 60% as compared to the level prior to treatment. Additionally, the plasma level of triglycerides may be decreased below at least 200 mg/DL or at least 150 mg/DL. Further, the method may comprise maintaining the plasma level of cholesterol below the level prior to the treatment by the percentages described above and/or below the values described above.

An additional aspect of the present invention concerns a method for the treatment of dislipoproteinemia in a human subject in need thereof comprising periodically orally administering to the human subject 3,3,14,14 tetramethyl hexadecane 1,16 dioic acid in a range from about 30 mg per day to about 800 mg per day.

An additional aspect of the present invention concerns a method for the treatment of hyperlipidemia in a human subject in need thereof comprising periodically orally administering to the human subject 3,3,14,14 tetramethyl hexadecane 1,16 dioic acid in a range from about 30 mg per day to about 800 mg per day.

The present invention further provides a method for the treatment of hypertension in a human subject in need thereof comprising periodically orally administering to the human subject 3,3,14,14 tetramethyl hexadecane 1,16 dioic acid in a range from about 30 mg per day to about 800 mg per day. The blood pressure may decrease to below at least 160 mmHg systolic and /or 100 mmHg diastolic, at least 140 mmHg systolic and/or 90 mmHg diastolic, or at least 120 mmHg systolic and/or 80 mmHg diastolic. Further, the method may comprise maintaining the blood pressure below these values.

An additional aspect of the present invention concerns a method of delaying the onset of non-insulin dependent diabetes mellitus in a human subject susceptible thereto comprising periodically orally administering to the human subject 3,3,14,14 tetramethyl hexadecane 1,16 dioic acid [in a range from about 30 mg per day to about 800 mg per day]. In one aspect, this method comprises decreasing the resistance to insulin. Insulin resistance may be measured using several methods, as described in Example 4. In another aspect, the plasma level of fasting glucose in the human subject is decreased, optionally below 126 mg/DL or 100 mg/DL. The method may further comprise maintaining the decreased insulin resistance or decreased plasma level of fasting glucose.

The dosage of 3,3,14,14 tetramethyl hexadecane 1,16 dioic acid according to any of the above methods as described herein may be from about 30 mg per day to about 600 mg per day; from about 30 mg per day to about 400 mg per day; from about 100 mg per day to about 600 mg per day; or from about 200 mg per day to about 400 mg per day. Additionally, the periodic administration of 3,3,14,14 tetramethyl hexadecane 1,16 dioic acid may be effected twice daily; three times daily; at least once daily for at least 14 days; at least once daily for at least 21 days; or at least once daily for at least 30 days.

The advantages of lower doses are evident to those of skill in the art. These include, inter alia, a lower risk of side effects, especially in long-term use, and a lower risk of the patient becoming desensitized to the treatment.

The treatment of different conditions may indicate the use of different doses or different time periods; these will be evident to the skilled medical practitioner. For example, lowering triglycerides, lowering LDL and/or VLDL cholesterol, lowering total cholesterol and elevating HDL cholesterol may be effected using doses of 3,3,14,14 tetramethyl hexadecane 1,16 dioic acid in the range of from about 30 mg per day to about 400 mg per day and/or may be effected following at least 14 days of treatment, while treatment of other adverse indications may be effected using doses of 3,3,14,14 tetramethyl hexadecane 1,16 dioic acid in the range of from about 100 mg per day to about 800 mg per day and/or may be effected following at least 30 days of treatment.

An additional aspect of the present invention concerns any of the above methods used in the treatment of a female subject, wherein the amount of 3,3,14,14 tetramethyl hexadecane 1,16 dioic acid is from about 30 mg per day to about 600 mg per day. This aspect also contemplates the treatment of a male subject, wherein the amount of 3,3,14,14 tetramethyl hexadecane 1,16 dioic acid is from about 100 per day to about 800 per day. Differences in the dosage regimen between males and females may also vary according to the condition to be treated, as described above; in general, animal results obtained by us indicate that females may have higher sensitivity to 3,3,14,14 tetramethyl hexadecane 1,16 dioic acid, thus suggesting the use of lower doses and/or less frequent administration and/or shorter time periods of treatment to humans.

It is to be understood that the minimal/maximal values of the various parameters to be measured, as indicated herein, may change from time to time according to the definitions and/or guidelines of the FDA.

While 3,3,14,14 tetramethyl hexadecane 1,16 dioic acid is primarily administered orally according to the methods of the present invention, other modes of administration are contemplated. For further detail regarding administration and formulation, see Example 5.

The invention has been described in an illustrative manner, and it is to be understood that the terminology which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

Throughout this application, various publications, including United States patents, are referenced by author and year and patents by number. The disclosures of these publications and patents and patent applications in their entireties are hereby incorporated by reference into this application in order to more fully describe the state of the art to which this invention pertains.

### EXAMPLES

Without further elaboration, it is believed that one skilled in the art can, using the preceding description, utilize the present invention to its fullest extent. The following preferred specific embodiments are, therefore, to be construed as merely illustrative, and not limitative of the claimed invention in any way.

Standard molecular biology protocols known in the art not specifically described herein are generally followed essentially as in Sambrook et al., Molecular cloning: A laboratory manual, Cold Springs Harbor Laboratory, New-York (1989, 1992), and in Ausubel et al., Current Protocols in Molecular Biology, John Wiley and Sons, Baltimore, Maryland (1988).

Standard organic synthesis protocols known in the art not specifically described herein are generally followed essentially as in Organic syntheses: Vol. 1-79, editors vary, J. Wiley, New York, (1941- 2003); Gewert et al., Organic synthesis workbook, Wiley-VCH, Weinheim (2000); Smith & March, Advanced Organic Chemistry, Wiley-Interscience; 5th edition (2001).

Standard medicinal chemistry methods known in the art not specifically described herein are generally followed essentially as in the series “Comprehensive Medicinal Chemistry”, by various authors and editors, published by Pergamon Press.

### Example 1

### Pharmacokinetics of Medica 16 in Rat and Dog 13-week Oral Toxicity Studies

The pharmacokinetics of Medica 16 were determined on day 10 of a repeated dose study in rats and dogs. These studies were conducted in parallel with 4-week oral GLP toxicity studies in each respective species. The steady state pharmacokinetic exposure parameters (Cmax, AUC₀₋₂₄) indicated that the extent of absorption in both species was non-linear, and the incremental increase of these parameters was less than the incremental increase of the dose. 13-week (89) oral GLP toxicity studies of Medica 16 were also conducted. Both studies included initial and steady state pharmacokinetic evaluations. As in the previous studies, both Cmax and AUC were non linear in both species as is shown in Table 1 (rats) and Table 2 (dogs).

TABLE 1
Mean dose-normalized pharmacokinetic exposure parameters*
of Medica 16 in rats during a 13-week oral toxicity study.

| | | | Dose, mg/kg/day | | | |
|---|---|---|---|---|---|---|
| Sex | Parameter | Study Day | 100 | 400 | 800 | 1600 |
| Males | Cmax, μg/mL | 1 | 156 | 58.2 | 44.5 | 21.1 |
| | | 30 | 240 | 95.2 | 64.1 | 28.2 |
| | | 90 | 174 | 74.5 | 51.6 | 26.6 |
| | AUC, μg · hr/mL | 1 | 1860 | 780 | 512 | 347 |
| | | 30 | 2365 | 1475 | 844 | 568 |
| | | 90 | 1630 | 832 | 631 | 349 |
| Females | Cmax, μg/mL | 1 | 184 | 94.2 | 60.0 | 28.1 |
| | | 30 | 285 | 122.0 | 55.5 | 39.5 |
| | | 90 | 231 | 81.0 | 50.4 | 0.27 |
| | AUC, μg · hr/mL | 1 | 2450 | 1210 | 710 | 463 |
| | | 30 | 3250 | 1862 | 941 | 656 |
| | | 90 | 2300 | 1082 | 612 | 407 |

*Parameters were dose-normalized to the 100 mg/kg/day dose. AUC₀₋₂₄

TABLE 2
Mean dose-normalized pharmacokinetic exposure parameters*
of Medica 16 in dogs during a 13-week oral toxicity study.

| | | | Dose, mg/kg/day | | | |
|---|---|---|---|---|---|---|
| Sex | Parameter | Study Day | 50 | 100 | 400 | 800 |
| Males | Cmax, μg/mL | 1 | 109 | 71.0 | 24.9 | 13.1 |
| | | 89 | 110 | 68.1 | 26.2 | 16.3 |
| | AUC, μg · hr/mL | 1 | 1252 | 810 | 347 | 188 |
| | | 89 | 1391 | 756 | 333 | 204 |
| Females | Cmax, μg/mL | 1 | 105 | 72.5 | 25.4 | 13.4 |
| | | 89 | 118 | 77.0 | 36.9 | 21.2 |
| | AUC, μg · hr/mL | 1 | 1292 | 894 | 391 | 187 |
| | | 89 | 1449 | 837 | 372 | 218 |

*Parameters were dose-normalized to the 50 mg/kg/day dose. AUC₀₋₂₄

### Example 2

### Single and Repeated Dose Oral Pharmacokinetics of Medica 16 in Healthy Male Volunteers

An open label, single center study of three groups was designed: single dose (Group 1), seven days of daily dosing (Group 2), and up to 27 days of daily dosing (Group 3).

The study was performed in 15 healthy male volunteers 25 to 52 years old. Body weight did not exceed 20% of ideal weight. The subjects were kept on an isocaloric diet consisting of 55% carbohydrate, 15% protein, and 30% fat (1:1 saturated: polyunsaturated fatty acids) and 350 mg/day cholesterol. The subjects were divided into three groups (2 subjects participated in more than one group) and administered M16 orally for up to 4 weeks.

| | | Dose | | |
|---|---|---|---|---|
| Group | N | (mg) | Treatment Duration | |
| 1 | 6 | 400 | 1 | day |
| 2 | 6 | 200 | 1 | day |
| | | 100 | 6 | days |
| 3 | 6 | 200 | 1 | day |
| | | 100 | 4 | weeks |

The test product used was Medica 16 in unformulated capsules (100 and 200 mg) of the free acid, administered orally in fasted state: single 400 mg dose (Group 1); initial single 200 mg dose, followed by 100 mg/day (Groups 2 and 3).

In the single 400 mg dose group (4.8 mg/kg), the mean plasma Cₘₐₓ was 28.7 μg/mL and was achieved at a mean Tₘₐₓ of 6.7 hours. The terminal elimination half-life was approximately 31 hours; Cl/F and V/F were suggestive of a relatively slow total body clearance and wide distribution, respectively.

The mean AUCO₀₋₇₂ was 1028 μg·hr/mL; the comparable AUC in male rats at 400 mg/kg/day (the NOAEL) was 5597 μg·hr/mL, and 6757 μg·hr/mL at 800 mg/kg/day in male dogs. This appears to indicate a wide safety margin in humans.

In the repeated-dose group, pre-dose plasma concentrations were taken at various days during the study. The data were highly variable, but a steady-state concentration of 20-40 μg/mL could be estimated.

Serial plasma samples, 0-72 hours, were obtained from Group 1, whereas pre dose samples were obtained from Groups 2 and 3. Concentrations of Medica 16 were quantitated by gas chromatography of the methyl esters; the LOQ was 0.5 μg/mL. Non-compartmental methods were used to determine the pharmacokinetic parameters (Cₘₐₓ, Tₘₐₓ, AUCₜ, AUC_{ing}, t_{1/2}, Cl/F and V/F) for Group 1.

Safety/tolerance evaluations included physical examinations, vital signs, ECG's, clinical chemistry, and hematology, urinalysis and ophthalmology.

### Results:

Medica 16 was well tolerated by all subjects with no changes in the safety parameters. The mean (±SD) pharmacokinetic parameters (Group 1) were as follows:

| Cₘₐₓ | Tₘₐₓ | AUCₜ | AUC_{inf} | T_{1/2} | Cl/F | V/F |
|---|---|---|---|---|---|---|
| (μg/mL) | (hr) | (μg · hr/mL) | | (hr) | (mL/hr/kg) | (L/kg) |
| 28.7 ± 12.7 | 6.7 ± 2.4 | 1028 ± 547 | 1320 ± 648 | 31.5 ± 12.8 | 4.5 ± 2.1 | 0.20 ± 0.09 |

In the repeated dose groups, the pre dose Medica 16 concentrations were highly variable over the course of the respective treatment periods. However, a steady-state concentration of 20-40 μg/mL can be estimated.

### Example 3

### A Pilot Safety, Efficacy and Pharmacokinetics Study of Medical 6 for Up to Four Months in Obese, Dyslipoproteinnemic Male Subjects

The safety and efficacy of Medica 16 (M16) was evaluated in eight male obese (BMI>28 kg/m²) dyslipoproteinemic (plasma triglycerides >300 mg/dL; HDL-cholesterol <35 mg/dL, normal or increased plasma cholesterol) nondiabetic subjects. Enrolled subjects reported failure of dietetic intervention aimed at both weight reduction and lowering of plasma lipids.

Each subject was given a 4-5 week placebo run-in prior to drug treatment with M16; M16 was then administered orally for a period of 3-4 months. Treatment was initiated at 200 mg qd and was gradually increased up to 800 mg qd (5 subjects received up to 400 mg, 2 subjected received up to 600 mg, and 1 subject received up to 800 mg). Upon termination of treatment, each patient was placed back on placebo for an additional month.

The enrolled subjects were placed on an isocaloric diet to maintain body weight and blood lipids and placed on a 4-5 month placebo treatment study run in. The starting dose for all subjects was 200 mg Medica 16 per day. One subject was maintained on 200 mg/day for three months. For the other subjects, after approximately 2 weeks at this dose, the dose was escalated step wise up to 300 mg/day (two subjects), 400 mg/day (two subjects), 600 mg/day (two subjects), and 800 mg/day (one subject). Safety, plasma triglycerides, cholesterol and Medica 16 concentrations were monitored throughout the study.

Triglyceride levels decreased (mean 46%) within the first month of treatment in all subjects and this was observed even at the lowest dose (200 mg/day); the overall mean decrease in triglycerides was 55%. The decline in plasma cholesterol was 13% in the first month and approximately 16% overall. Concomitant with the decrease in cholesterol, there was a mean increase of 12.6% in HDL in 5 of 8 subjects (range: 8%-19%); the increase was 46% in one of the 8 subjects. Plasma concentrations of Medica 16 were obtained in 6 of the 8 subjects. The data was variable, probably reflecting the dosing regimen. The incremental increase in concentrations was lower than the incremental increase in dose.

One subject experienced elevated CPK in week 10 of the study; levels returned to normal without a change/stoppage of his Medica 16 dosage. There were no other changes in clinical chemistry or hematology values and no adverse events were reported.

### Example 4

### Preliminary Results of Pilot Efficacy Study of Medica 16 (M16) for Up to 32 Weeks in Obese, Dyslipoproteinemic, Insulin Resistant Male Subjects

The hypolipidemic—calorigenic—anti—diabetic influence of M16 was examined in subjects suffering from Metabolic Syndrome. Each participant started the trial with the ingestion of a placebo, transferred to increasing doses of M16, and ended with a placebo. During the experiment, each participant underwent various tests both for safety and efficacy of the drug. Each participant was treated with a placebo for 5 weeks, switched to increasing doses of M16—at least 3 doses per participant, each dose for at least 4 weeks. At the end of the treatment, each participant was again treated with a placebo for at least 6 weeks.

The first 3 participants received 200, 400 or 600 mg M16; The remaining 2 participants received 30, 100, or 200 mg M16.

Hypolipidemic effect of M16:

Summary of changes in plasma levels of TG, Cholesterol, LDL-C, HDL-C and VLDL-C effecting response to M16:

| | Average change after treatment with M16 (%) |
|---|---|
| Triglycerides | −36.5 ± 8.0# |
| Cholesterol | −5.3 ± 5.6 |
| LDL-C | −1.6 ± 4.0 |
| HDL-C | +8.4 ± 6.2 |
| VLDL-C | −34.4 ± 11.3 |

### Calorigenic Effect of M16:

a. No significant change in body weight of participants was observed as a result of M16 treatment.
b. Treatment with M16 did not effect the participants' body composition, as reflected by a lack of significant change in both lean body mass and in body fat percent.
c. Following treatment, mild fluctuations in total energetic expenditure were observed, but with no statistically significant change.
   Anti—diabetic effects of M16:
a. M16 treatment in IGT (Impaired Glucose Tolerance) participants accelerates plasma insulin clearance
b. M16 treatment in IGT participants induces hepatic sensitivity to insulin, which is expressed by a decrease in the HOMA index.
c. M16 treatment in IGT participants induces peripheral sensitivity to insulin, which is expressed by an increase in K Glucose and more clearly in the Sr.
d. M16 treatment in IGT participants at high to supra-physiological insulin levels does not influence peripheral insulin sensitivity.

The drug causes insulin sensitization in IGT conditions.

### Example 5

### Methods of Measuring Insulin Resistance

| Test | Step 1 | Step 2 | Step 3 | Index | Interpretation |
|---|---|---|---|---|---|
| Hyperinsulinemic | Insulin | Glucose 20% | Insulin | Average GIR | Under this steady |
| Euglycemic | infusion | titration until | termination | during the | state if euglycemia: |
| Clamp | for 180 | GIR is stable | after 180 min | final 30 min of | Glucose infusion |
| | min. | (~45 min) and | Glucose | the test (150- | rate equals glucose |
| | | euglycemia is | infusion | 180 min) | uptake by all tissues |
| | | achieved | continues until | | and therefore a |
| | | | euglycemia is | | measure if tissue |
| | | | maintained on | | sensitivity t0 |
| | | | its own | | exogenous insulin |
| Hyperglycemic | Glucose | The desired | | | Under constant |
| clamp | infusion | hyperglycemic | | | hyperglycemia: |
| | titration: | plateau is | | | Plasma insulin |
| | plasma | subsequently | | | response is biphasic |
| | glucose | maintained by | | | with an early burst |
| | level is | adjustment of | | | (6 min) followed by |
| | raised to | the glucose | | | a gradually |
| | 125 mg % | infusion | | | progressive |
| | above | | | | increase in plasma |
| | basal level | | | | insulin |
| | | | | | concentration (beta |
| | | | | | cell function to the |
| | | | | | fasting |
| | | | | | hyperglycemia) |
| HOMA-IR* | | | | [Fasting | Quantitative |
| Homeostasis | | | | insulin | assessment of the |
| model assessment | | | | (uU/mL) × | contributions of |
| of insulin | | | | fasting | insulin and |
| resistance index | | | | glucose | deficient beta-cell |
| | | | | (mg %)]/405 | function to the |
| | | | | | fasting |
| | | | | | hyperglycemia |
| HOMA-B %** | | | | 20 × fasting | Indices of |
| | | | | insulin in | pancreatic beta-cell |
| | | | | mU/L (fasting | function |
| | | | | glucose in | |
| | | | | mmol/1-3.5) | |
| QUICKI*** | | | | 1/[log fasting | |
| Quantitative | | | | insulin | |
| insulin sensitivity | | | | (uU/mL) + log | |
| check index | | | | glucose | |
| | | | | (mg/dL) | |
| Insulinogenic | | | | Ratio of | Indices of |
| index**** | | | | fasting insulin | pancreatic beta-cell |
| | | | | (uU/mL) and | function |
| | | | | fasting | |
| | | | | glucose | |
| | | | | (mg/dL) | |

*correlated with estimates obtained by use of the euglycemic clamp (Rs = 0.88, p < 0.0001), the fasting insulin concentration (Rs = 0.81, p < 0.01) - (Matthews et al, Diabetologia 1985).
**the estimate of deficient beta-cell function obtained by homeostasis model assessment correlated with that derived using the hyperglycemic clamp (Rs = 0.61, p < 0.01) and with the estimate from the intravenous glucose tolerance test (Rs = 0.64, p < 0.05) (see reference above).
***Quantitative insulin sensitivity checka index (QUICKI) is correlated with SI (Clamp) (r = 0.78) (Katz et al., J Clin Endocrinol Metab 2000).
****FIG ratio was directly correlated with the HOMA index (r = 0.83, p < 0.01) and fasting insulin, (r = 0.95, p < 0.001) (Guerrero-Romero, Diabetes Metab 2001).

### Example 6

### Formulation

Medica 16 is administered orally according to the above described dosages. It should be noted that the compound can be administered as the compound or as a pharmaceutically acceptable salt and can be administered alone or as an active ingredient in combination with pharmaceutically acceptable carriers, solvents, diluents, excipients, adjuvants and vehicles. M16 is preferably administered orally, but can also be administered subcutaneously or parenterally including intravenous, intraarterial, intramuscular, intraperitoneally, and intranasal administration as well as intrathecal and infusion techniques. Implants of M16 are also useful. Liquid forms may be prepared for injection, the term including subcutaneous, transdermal, intravenous, intramuscular, intrathecal, and other parental routes of administration. The liquid compositions include aqueous solutions, with and without organic cosolvents, aqueous or oil suspensions, emulsions with edible oils, as well as similar pharmaceutical vehicles. In addition, under certain circumstances M16 may be formed a an aerosol, for intranasal and like administration. The pharmaceutically acceptable carriers, solvents, diluents, excipients, adjuvants and vehicles as well as implant carriers generally refer to inert, non-toxic solid or liquid fillers, diluents or encapsulating material not reacting with the active ingredients of the invention.

When administering M16 parenterally, it is generally formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, can also be used as solvent systems for compound compositions. Additionally, various additives which enhance the stability, sterility, and isotonicity of the M16 compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it is desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used have to be compatible with M16.

Sterile injectable solutions can be prepared by incorporating the compounds utilized in practicing the present invention in the required amount of the appropriate solvent with various of the other ingredients, as desired.

A pharmacological formulation of M16 can be administered to the patient in an injectable formulation containing any compatible carrier, such as various vehicle, adjuvants, additives, and diluents; optionally, M16 can be administered parenterally to the patient in the form of slow-release subcutaneous implants or targeted delivery systems such as monoclonal antibodies, vectored delivery, iontophoretic, polymer matrices, liposomes, and microspheres. Examples of delivery systems useful in the present invention include U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

A pharmacological formulation of M16 utilized in the present invention can be administered orally to the patient. Conventional methods such as administering the compound in tablets, suspensions, solutions, emulsions, capsules, powders, syrups and the like are usable. Known techniques which deliver it orally or intravenously and retain the biological activity are preferred. In one embodiment, M16 can be administered initially by intravenous injection to bring blood levels to a suitable level. The patient's levels are then maintained by an oral dosage form, although other forms of administration, dependent upon the patient's condition and as indicated above, can be used.

1. A method for treatment of a symptom associated with Metabolic Syndrome in a human subject in need thereof comprising periodically orally administering to the human subject 3,3,14,14 tetramethyl hexadecane 1,16 dioic acid in an amount from about from about 30 mg per day to about 800 mg per day.

2. A method for elevating the plasma level of HDL cholesterol in a human subject in need thereof comprising periodically orally administering to the human subject 3,3,14,14 tetramethyl hexadecane 1,16 dioic acid in a range from about 30 mg per day to about 800 mg per day.

3. The method of claim 2 wherein the plasma level of HDL cholesterol is elevated by at least 5% as compared to the level prior to treatment and/or above at least 30 mg/DL.

4. The method of claim 3 further comprising maintaining the plasma level of HDL cholesterol at least 5% above the level prior to the treatment and/or above at least 30 mg/DL.

5. A method for decreasing the plasma level of LDL cholesterol in a human subject in need thereof comprising periodically orally administering to the human subject 3,3,14,14 tetramethyl hexadecane 1,16 dioic acid in a range from about 30 mg per day to about 800 mg per day.

6. The method of claim 5 wherein the plasma level of LDL cholesterol is decreased by at least 10% as compared to the level prior to treatment and/or below at least 190 mg/DL.

7. The method of claim 6 further comprising maintaining the plasma level of LDL cholesterol at least 10% below the level prior to treatment and/or below at least 190 mg/DL.

8. A method for decreasing the plasma level of triglycerides in a human subject in need thereof comprising periodically orally administering to a human subject 3,3,14,14 tetramethyl hexadecane 1,16 dioic acid in a range from about 30 mg per day to about 800 mg per day.

9. The method of claim 8 wherein the plasma level of triglycerides is decreased by at least 10% as compared to the level prior to treatment and/or below at least 200 mg/DL.

10. The method of claim 9 further comprising maintaining the plasma level of triglycerides at least 10% below the level prior to treatment and/or below at least 200 mg/DL.

11. A method for the treatment of dislipoproteinemia in a human subject in need thereof comprising periodically orally administering to the human subject 3,3,14,14 tetramethyl hexadecane 1,16 dioic acid in a range from about 30 mg per day to about 800 mg per day.

12. A method for the treatment of hyperlipidemia in a human subject in need thereof comprising periodically orally administering to the human subject 3,3,14,14 tetramethyl hexadecane 1,16 dioic acid in a range from about 30 mg per day to about 800 mg per day.

13. A method for the treatment of hypertension in a human subject in need thereof comprising periodically orally administering to the human subject 3,3,14,14 tetramethyl hexadecane 1,16 dioic acid in a range from about 30 mg per day to about 800 mg per day.

14. The method of claim 13 wherein the cystolic and/or diastolic blood pressure is decreased to below at least 160 mmHg systolic and /or 100 mmHg diastolic.

15. The method of claim 14 further comprising maintaining the blood pressure below at least 160 mmnHg systolic and /or 100 mmHg diastolic.

16. A method of delaying the onset of non-insulin dependent diabetes mellitus in a human subject susceptible thereto comprising periodically orally administering to the human subject 3,3,14,14 tetramethyl hexadecane 1,16 dioic acid in a range from about 30 mg per day to about 800 mg per day.

17. The method of claim 16 whereby the insulin resistance is decreased.

18. The method of claim 16 whereby the plasma level of fasting glucose is decreased to below at least 126 mg/DL.

19. The method of claim 18 further comprising maintaining the plasma level below at least 126 mg/DL.

20. The method of claim 1 wherein the amount is from about 30mg per day I to about 600 mg per day.

21. The method of claim 1 wherein the amount is from about 30 mg per day to about 400 mg per day.

22. The method of claim 1 wherein the amount is from about 100 mg per day to about 600 mg per day.

23. The method of claim 1 wherein the amount is from about 200 mg per day I to about 400 mg per day.

24. The method of claim 1 wherein the human subject is a female and the amount is from about 30 mg per day to about 600 mg per day.

25. The method of claim 1 wherein the periodic administration is effected twice daily.

26. The method of claim 1 wherein the periodic administration is effected three times daily.

27. The methods of claim 1 wherein the periodic administration is effected at least once daily for at least 14 days.

28. The methods of claim 1 wherein the periodic administration is effected at least once daily for at least 21 days.

29. The methods of claim 1 wherein the periodic administration is effected at least once daily for at least 30 days.
